(19) United States
(12) Patent Application Publication
(10) Pub. No.: US 2025/0060366 A1
(43) Pub. Date: Feb. 20, 2025

(54) IMMUNOCHROMATOGRAPHIC TEST STRIP WITH MULTIPLE FLOW PATHS, AND MANUFACTURING METHOD THEREFOR

(71) Applicant: TECHNISCHE UNIVERSITÄT BRAUNSCHWEIG, Braunschweig (DE)

(72) Inventors: Andreas Dietzel, Braunschweig (DE); Monika Leester-Schädel, Braunschweig (DE); Esteban Builes-Münden, Braunschweig (DE); Günther Gauglitz, Tübingen (DE); Günther Proll, Tübingen (DE); Monika Conrad, Tübingen (DE); Johanna Hutterer, Tübingen (DE)

(21) Appl. No.: 18/721,820

(22) PCT Filed: Dec. 21, 2022
(86) PCT No.: PCT/EP2022/087252

§ 371 Date: Jul. 9, 2024

(30) Foreign Application Priority Data
Dec. 21, 2021 (DE) 10 2021 214 853.0

(51) Int. Cl. G01N 33/543; B01L 3/00; G01N 33/58

(52) U.S. Cl. CPC G01N 33/54389; B01L 3/5023; G01N 33/54393; G01N 33/58; B01L 2200/16; B01L 2300/0825; B01L 2400/0406; B01L 2400/0677; B01L 2400/0688

(57) ABSTRACT

A test strip for analysing samples, in particular blood or serum samples, characterised by at least two, preferably parallel, flow paths for liquid, which are accessible from a common feed material onto which a sample can be applied. The flow paths can consist of porous material, in particular a nitrocellulose layer, on a carrier that is impermeable to aqueous liquid, e.g. plastic film or hydrophobised paper. The flow paths are spaced apart from each other, wherein this spacing can optionally be filled with hydrophobic material.

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2022/087252, which was filed Dec. 21, 2022, which application claimed priority from German Application 102021214853.0, which was filed Dec. 21, 2021.

### FIELD OF THE INVENTION

A field of the invention concerns test strip for the analysis of samples. Test strips of the invention have example applications to the analysis of particular of blood or serum samples, e.g. obtainable by centrifugation of whole blood, urine or saliva samples, or analytes dispersed in water.

### BACKGROUND

In contrast to the present invention, test strips for so-called sandwich analyses on an analyte require at least two epitopes so that a capture antibody can bind to one of the epitopes and a labelled antibody can also bind to another epitope of the same analyte.

Hecht et al, Microelectronic Engineering 158, 52-58 (2016) describes the fabrication of parallel hydrophilic paths from a nitrocellulose layer by laser ablation of nitrocellulose between the desired paths, whereby multiple laser treatments along the ablated areas essentially close the adjacent edges of the paths of nitrocellulose by fusion and prevent leakage of liquid into the ablated area. The paths can therefore form parallel channels with closed surfaces on the long sides, between which the transfer of liquid is prevented.

EP 3 171 169 B1 describes the production of parallel separated paths in e.g. a nitrocellulose layer applied to a carrier by removing nitrocellulose from the underlying carrier by laser irradiation, e.g. an Nd:YVO₄ laser with a wavelength of 532 nm, a pulse duration of 12 ps, a pulse energy of 10 mJ and a pulse frequency of 10 KHz.

### SUMMARY OF THE INVENTION

Preferred embodiments provide a test strip and an analytical method which can be carried out with it, in particular for use in the detection of analytes which have exactly one epitope. The test strip enables simultaneous contacting of at least two separate flow paths with exactly one feed material for a sample, whereby the inflow of the sample into the flow paths is controllable after an incubation period. Preferably, the test strip and the analytical method are suitable for the quantitative detection of an analyte or for the parallel detection of different analytes.

### BRIEF DESCRIPTION OF THE DRAWINGS

The invention will now be described in more detail by means of examples and with reference to the figures which show in

FIG. 1 a schematic view of an embodiment of the test strip,

FIG. 2A to C a schematic of an embodiment of the test strip in the analytical process,

FIG. 3A scanning electron micrograph of a cross-section through a test strip in the area of the surface of the long side of a flow path,

FIG. 4A scanning electron micrograph of the surface of the long side of a flow path made of nitrocellulose on a carrier,

FIG. 5A-C a scanning laser microscopic analysis of the recess of a flow path and

FIG. 6 the evaluation of the analyte content for six different amounts of analyte, each applied in one compartment.

### DETAILED DESCRIPTION OF THE PREFERRED EMBODIMENTS

The present invention relates to a test strip for the analysis of samples, in particular of blood or serum samples, e.g. obtainable by centrifugation of whole blood, urine or saliva samples, or analytes dispersed in water, which test strip is characterised by at least two, preferably parallel, flow paths for liquid which are accessible from a common feed material onto which a sample can be applied. The flow paths consist of porous material, in particular a nitrocellulose layer, on a carrier that is impermeable to aqueous liquid, e.g. plastic film or hydrophobised paper. The flow paths are spaced apart from each other, wherein this space can optionally be filled with hydrophobic material. No porous hydrophilic material permeable to liquid is present in the space between the flow paths, preferably no nitrocellulose. Preferably, the porous material in the space between flow paths has been removed, in particular by laser irradiation. Preferably, the surfaces of the longitudinal sides of the flow paths are impermeable to liquid, in particular these surfaces consist of a thin continuous layer consisting of previously porous material melted and resolidified in the laser process, e.g. a portion of the original porous material melted and resolidified by laser irradiation.

The test strip is characterised by the flow paths being set up so that after an incubation period they can be connected simultaneously to the feed material and/or a reagent reservoir connected to it. Therefore, a sample applied to the feed material can distribute over the feed material and/or a reagent reservoir connected to it and the sample can only flow into the flow paths once a barrier in the flow paths has been overcome. The barrier has the advantage that the sample is incubated with a binding molecule specific for an analyte, optionally additionally with spiked analyte and/or a competitor of the analyte, before flowing into the flow paths and an equilibrium of the analytes bound to the binding molecule, optionally with spiked analyte and/or a competitor of the analyte, is established. The duration of the incubation can be set by means of the barrier, preferably the barrier is set up to open abruptly or within a short time in order to connect the feed material with all flow paths simultaneously. The incubation time predetermined by the barrier leads to an increased accuracy of the detection, in particular the quantification of the analyte that was applied with the sample.

Preferably, the feed material and/or the reagent reservoir connected to it is provided with the binding molecule with which the added sample can mix and react, optionally additionally with spiked analyte and/or a competitor of the analyte, before the mixture thus obtained can flow into the flow paths after opening or overcoming the barrier.

The test strip and the analytical method according to the invention are particularly suitable for use in quantitative analyses, since the at least two flow paths permit quantification of an analyte. The test strip is suitable for use in analysing an analyte by means of only one antibody, wherein the analyte preferably has exactly one epitope to which an antibody can bind, or the analyte only allows the binding of exactly one antibody molecule and prevents the binding of another antibody molecule, for example for spatial reasons. Therefore, the test strip and the method of analysis which can be carried out with it have the advantage of also being suitable for use in analysing an analyte which has exactly one epitope. The invention also relates to a method for producing the test strip.

The invention achieves the object by the features of the claims and, in particular, provides a test strip, and its use in the analytical method, which comprises or constists of
at least two flow paths made of liquid-permeable porous material, which are mounted on a common carrier,
wherein the flow paths are connected to a feed material at their first end,
wherein preferably the flow paths are in contact with a suction material at their second end opposite the first end,
wherein a reagent reservoir is disposed between the feed material and the flow paths and in contact with their first ends, the reagent reservoir being divided at its second end into compartments, each of which is connected to exactly one flow path at its first end, and the compartments being connected to the feed material at their opposite first end by means of a common portion of the reagent reservoir,
optionally a filter layer arranged between the feed material and the reagent reservoir or between the reagent reservoir and adjacent to the flow paths,
each of the flow paths having a detection area between its first end and its second end,
optionally, each of the flow paths having a control area between its detection area and its second end,
wherein a barrier is arranged between the second end of the reagent reservoir, in particular the compartments, and each of the flow paths.

The barrier arranged between the second end of the reagent reservoir, or the second ends of the compartments of the reagent reservoir, and the flow paths serve to incubate the mixture of sample, which has flowed through the feed material into the reagent reservoir, and binding molecule, which is contained in the reagent reservoir or in the feed material, optionally additionally analyte and/or competitor of the analyte as spiking, before the barrier is opened, and to connect the mixture to the flow paths after opening the barrier, preferably all at the same time. Preferably therein, the compartments of the reagent reservoir contain different amounts of the analyte and/or of the analyte's competitor contained as a spike, and/or different binding molecules that are specific for different analytes, and/or different amounts of the binding molecule.

The at least two flow paths allow for the parallel determination of the analyte with at least one or two amounts of analyte added to the sample to spike the analyte. Alternatively or additionally, the at least two flow paths allow for parallel analysis with at least two different binding molecules that are specific for the same analyte or for different analytes.

The first end of the reagent reservoir is connected to the feed material so that sample applied to the feed material flows into the reagent reservoir. In a section adjacent to the first end, the reagent reservoir is divided into parallel compartments, which are preferably created by laser ablation, so that the sample flows into the compartments. In each of the compartments, the sample is brought into contact with the binding molecule, optionally additionally with spiked analyte and/or competitor of the analyte, a different binding molecule and/or a different amount of the same binding molecule and incubated in one of the compartments until the barrier is opened. The compartments are preferably parallel and spaced-apart strips which are formed in one piece with the section connecting them at the first end.

Preferably, the first end of the reagent reservoir lies between the feed material, e.g. its second end, and the carrier, while the second end of the reagent reservoir, in particular the second ends of its compartments, lie on the first ends of the flow paths, opposite the carrier on which the flow paths are arranged.

The barrier can be arranged in the compartments of the reagent reservoir, for example at the second ends thereof, which are connected to the first ends of the flow paths. Preferably, the barrier is arranged in sections of the flow paths adjacent to their first ends.

The barrier can, for example, be embodied as a
sugar (e.g. sucrose) that has been applied as an aqueous sugar solution and dried so that the barrier is opened by dissolving the sugar,
wax, printed as a liquid, which is overcome by the addition of a detergent, e.g. Tween20, contained in the feed material and/or in the reagent reservoir,
salt, applied as an aqueous solution and dried so that the barrier is opened by dissolving the salt
PVA (polyvinyl alcohol), a water-soluble polymer that opens the barrier by dissolving on contact with the sample, e.g. dripped in solution onto a nitrocellulose membrane and then dried, as a hydrophobic barrier,
optionally, an additional glass fibre mat inserted into a recess which reduces the flow velocity,
an additional section of cellulose which is inserted into a recess so that at least part of the liquid flows through this cellulose, resulting in a delay,
spiropyran-doped poly (DEAEMA-co-MMA), which is hydrophobic and only becomes hydrophilic under UV irradiation, for photo-induced polarity change,
recess, e.g. produced by laser radiation, which prevents the flow and overcomes the gap formed by the recess by applying an electric field (the electric field reduces the contact angle between the sample and the hydrophobic carrier, e.g. made of polyester, and allows the sample to continue flowing, “electrowetting”),
light-switchable surfactant, e.g. donor-acceptor stenhouse adducts (DASA), which are molecules whose polarity, or whose hydrophobic or hydrophilic property, can be changed by visible light. In a preferred embodiment, preferably wax is applied as a barrier and a light-switchable surfactant is applied between this wax barrier and the feed material and covered with a light-tight cover, which is removed after the duration of an incubation or at a time interval after application of a sample to the feed material in order to allow light to act on the surfactant. The light can be daylight or come from a current-driven light source. The light-switchable surfactant becomes surface-active after exposure to light and leads to the aqueous sample overcoming the wax barrier. Alternatively, DASA molecules may be deposited in a laser-generated recess, where the DASA molecules are initially protected from light by a shield, and after the incubation period, the shield is removed and the DASA molecules are illuminated to convert them from a hydrophobic form to the surfactant surface-active form, allowing the sample to continue flowing.

In a preferred embodiment, the barrier is formed by a recess which extends over the complete cross-section of the compartment of the reagent reservoir and/or of the flow path, wherein the recess preferably extends into the carrier, or the carrier has a recess adjacent to the recess of the flow paths. Preferably, each of the flow paths, between its first end and its detection area has a recess which extends over the complete cross-section of the flow path or of a compartment of the reagent reservoir, the recess preferably extending into the carrier, or the carrier having a recess adjacent to the recess of the flow paths.

The recess which is present in each flow path, the recess preferably extending into the adjacent carrier, forms a barrier against liquid penetrating into the first end of the flow paths, for example from the feed material and/or from the reagent reservoir and/or from a filter layer. Therefore, the recess allows the incubation of a sample placed on the feed material in the feed material and/or in a reagent reservoir in contact therewith, and the subsequent controlled overcoming of the recess forming the barrier. The recess can be overcome by bending the carrier along the recess in order to bring the sections of a flow path spaced apart by the recess into contact with each other. It has been shown that bending the carrier along the recess until the sections spaced apart by the recess come into contact leads to the flow of liquid across the recess, and that this flow also remains when the carrier is bent back, e.g. when the carrier is bent back towards a plane. At least in the case of aqueous samples, e.g. blood serum, urine, saliva, water samples or analytes dispersed in water, the surface tension is sufficient to maintain the flow of liquid across the recess even after the carrier has been bent back towards a plane so that the recess extends over its original cross-section. These recesses of each flow path are preferably arranged at the same distance from the respective detection area, e.g. along a line that extends perpendicular to the longitudinal extension of the flow paths.

The recess in each of the flow paths extends over the entire cross-section of the porous material of the flow path. The recess of each flow path preferably has the same clear cross-section, in particular V-shaped from the surface of the porous material opposite the carrier, tapering towards the carrier or rectangular. Preferably, the surfaces of each flow path, which delimit the recess, are positioned at a distance from one another when the carrier extends in a plane and are brought into contact with one another by bending the carrier along the recess, wherein optionally the carrier is then bent back in the direction of a plane.

The recess is dimensioned such that when the carrier is arranged or aligned in a plane and before the carrier is bent around the recess towards the porous material, no liquid can extend beyond the recess, and that when the carrier is bent around the recess, the surfaces of each flow path delimiting the recess contact each other. The recess preferably has a cross-section which, in the plane of the carrier, has a distance between the opposing surfaces of at least 50 μm, more preferably of at least 100 or at least 200 μm. The recess can extend, for example, between the surfaces of each flow path delimiting the recess over 20 to 300 μm, e.g. 20 to 40 μm in the plane of the carrier, e.g. up to 150 to 300 μm, e.g. 200 to 250 μm in the plane of the surface of the porous material opposite the carrier. Preferably, the recess continues into the carrier, so that the carrier is thinner adjacent to the recess of the flow paths and forms a bending joint there. The recess can, for example, extend into the carrier from 10 to 80%, preferably 30 to 70% or up to 50% of the thickness of the carrier.

In a preferred method of manufacture, the recesses that form a barrier are created by removing the porous material by laser irradiation. For nitrocellulose, pulsed laser radiation of a wavelength of 1030 nm at a repetition rate of 600 kHz and a pulse duration of 212 ps, a movement of the laser beam over the porous material at 2400 mm/s, preferably with 15 to 20 repetitions along the area to be removed, at a pulse energy of 9.42 to 11.57 μJ, e.g. with a laser power of 5.655 W can be used for removal along the area to be removed for the recess. For nitrocellulose with a layer height of 160 to 200 μm on a transparent plastic as carrier, a recess can be created with a distance of approx. 70 to 85 μm between opposing surfaces at the centre height of the layer. Such a recess has a V-shaped cross-section that tapers towards the carrier.

The surfaces of each flow path that delimit the recess are open-pored.

The porous material can be open-pored nylon, cellulose acetate, polyethersulfone, cross-linked polydextran, preferably nitrocellulose. The carrier can consist of polyester, polypropylene, polyethylene, acrylic polymer, polycarbonate or hydrophobised paper. In the manufacturing process, the longitudinal sides of the flow paths are preferably produced with closed surfaces by treating the porous material along the surfaces of the longitudinal sides of the flow paths with laser radiation. Pulsed laser radiation of a wavelength of 1030 nm at a repetition rate of 600 kHz with a movement along the surfaces of the longitudinal sides at 2400 mm/s at a pulse energy of 11 to 12 μJ with at least 3 repetitions along the surfaces of the longitudinal sides can be used to produce the closed surfaces along the surfaces of the longitudinal sides. This laser radiation is preferably generated by the same laser that is used to generate the laser radiation for removing the material of the recess.

The flow paths preferably have the same layer height on the common carrier, e.g. with a layer height of 100 to 300 μm, preferably 120 to 270 μm, e.g. 120 to 160 μm or 240 to 270 μm. The porous material is preferably nitrocellulose, preferably with a nominal exclusion size of 0.22 to 0.45 μm.

The feed material may be directly in contact with the first ends of the flow paths or indirectly in contact with the first ends of the flow paths, e.g. through a reagent reservoir and/or a filter layer arranged between the feed material and the flow paths. Preferably, the reagent reservoir contains a binding molecule that is specific for the analyte, e.g. an antibody directed against the analyte.

The feed material, the reagent reservoir and an absorbent material can each independently be porous cellulose, glass wool or a mixture of these.

Optionally, a second reagent reservoir is arranged between the feed material and the reagent reservoir or between the reagent reservoir and adjacent to the flow paths. Preferably, a second reagent reservoir each is formed by the sections of the flow paths extending between the first end of the flow path, e.g. between the feed material, filter layer or reagent reservoir adjacent to the flow path on the one hand, and the recess on the other hand.

Such a second reagent reservoir may contain a competitive antagonist for the analyte, generally also referred to as a competitor, preferably second reagent reservoirs contain predetermined different amounts of the same competitive antagonist and/or different competitive antagonists and thus allow a spiking procedure. Optionally, a second reagent reservoir of a flow path may contain the analyte, optionally in admixture with a competitive antagonist thereof, to form an internal positive control for the analyte in the test strip.

A competitive antagonist of the analyte can, for example, be applied to a section of the flow paths by pressurisation using a nozzle. A second reagent reservoir of each flow path preferably extends between the first end of the flow path and the recess. A test strip with at least two flow paths, the sections of which form a second reagent reservoir between their first end and the recess which forms a barrier, preferably has different amounts of the same competitive antagonist and/or different competitive antagonists for the analyte in each of the second reagent reservoirs. Such test strips are set up for a quantitative analysis of the analyte, since the binding molecule is bound by the competitive antagonist depending on the amount of the latter and binds inversely proportional to the amount of the antagonist in the detection area.

The detection area preferably has an immobilised molecule that binds to the binding molecule like the analyte and preferably binds like the optional competitive antagonist. The detection area is therefore set up to bind binding molecules that are not bound to the analyte or to the optional competitive antagonist. The immobilised molecule in the detection area can be, for example, bound analyte, e.g. analyte bound to a carrier molecule.

Further preferably, each flow path between the detection area and its second end has a control area which is adapted to bind binding molecules independently of the presence of an analyte or of the optional competitive antagonist. In the preferred embodiment, the binding molecule is a labelled antibody and the control area comprises a second antibody specific for the constant heavy chain of the labelled antibody. Such a second antibody, preferably immobilised in the control area, serves as a positive control for the flow of liquid from the feed material, through the reagent reservoir, along the flow paths across the recess and the detection area.

The feed material can be multi-layered and optionally have a filter layer that is set up, for example, to retain cells from a whole blood sample.

In general, the feed material can be formed in one piece with the reagent reservoir.

In the manufacturing process, it is preferred that
a porous material arranged on a carrier, in particular fixed to it, is divided by laser irradiation into at least two flow paths separated from each other by a distance,
wherein preferably their surfaces of the longitudinal sides are fused from a first end to an opposite second end to form a liquid-impermeable surface,
a recess is created between the first end and the second end of each flow path by laser irradiation, which recess extends over the entire cross-section of the flow path and is delimited by opposing open-pored surfaces of the flow paths, wherein the recess preferably continues into the carrier,
a detection area is created between the recess and the second end of each flow path by applying an immobilised molecule that binds to the binding molecule like the analyte and preferably binds like the optional competitive antagonist,
optionally, a control area is created between the detection area and the second end of each flow path by applying an immobilised second antibody directed against the labelled binding molecule,
optionally, a second reagent reservoir is produced between the first end and the recess of at least one, preferably at least four, of the flow paths by applying the analyte and/or a competitive antagonist of the analyte,
a reagent reservoir containing a labelled binding molecule is placed in contact with the first end of each of the flow paths,
a feed material is arranged in contact with the reagent reservoir and opposite the flow paths,
optionally a suction material is arranged adjacent to the second ends of the flow paths,
wherein preferably, subsequent to the creation of the detection area, the optional control area and optional second reagent reservoirs, binding sites of the flow paths, optionally additionally of the feed area and/or of the reagent reservoir, are saturated, e.g. by contacting with a non-specific protein, e.g. serum albumin or casein.

In the analytical method, a test strip according to the invention is provided and a sample is applied to the feed material, preferably while the test strip is arranged in a plane. The sample flows by capillary forces through the reagent reservoir, through optional second reagent reservoirs up to adjacent to the barrier. After an incubation time, which may be e.g. 1 to 10 min, e.g. 5 min, the barrier is opened so that the sample can enter the at least two flow paths. In the embodiment in which the barrier is a recess, the barrier is opened by bending the test strip around the recess until the surfaces of the flow paths delimiting the recess touch and, as a result, liquid flows across the recess towards the second end of the flow paths. Optionally, the test strip can be bent back towards the original plane, as a film of liquid extends over the recess and allows the liquid to flow.

Until the barrier is opened, an equilibrium of the sample with the binding molecule and optionally with the added analyte and/or a competitor of the analyte can be established, so that the quantification of analytes contained in the original sample is more accurate, because the proportion of binding molecule that has not bound to analytes of the sample or added analyte or its competitor and therefore binds in the detection area is the equilibrium concentration, resp. in the sample, optionally with spiking, the minimum free amount of the binding molecule. After the incubation time, which is determined by the opening of the barrier, the liquid contains at least a portion of the sample and the labelled binding molecule from the reagent reservoir, as well as optionally spiked analyte and/or competitive antagonist from a compartment of the reagent reservoir and/or from one of the optional second reagent reservoirs. This liquid flows along the detection area in which unbound labelled binding molecule is bound and immobilised by the immobilised molecule, which binds to the binding molecule like the analyte and preferably like the optional competitive antagonist. The labelled binding molecule bound in the detection area is detected on the basis of the label, e.g. by optical measurement of the intensity of the colouring in the detection area. Therein the test strip has the advantage that the equilibrium between the binding molecule and the analyte, which has established in front of the barrier, is maintained or not disturbed in the detection area. Labelled binding molecules bound to analytes or to competitive antagonists are moved further along the flow path across the detection area and are bound and immobilised in the optional control area by an immobilised second antibody. The binding molecule bound in the control area is determined, for example, by optical measurement of the intensity of the colouring in the control area. Generally, a labelled binding molecule has a detectable label associated with the binding molecule, preferably an optically detectable label, e.g. a nanoparticle of metal, in particular a gold nanoparticle, or metal oxide, or a dye.

Preferably, in order to obtain a quantitative analysis of the analyte results of the measurements in the detection area and optionally in the control area are put in relation to the amount of spiked analyte and/or of competitive antagonist in the reagent reservoir, in particular its compartments, or in optional second reagent reservoirs.

Preferably, two, three, four or more of the second reagent reservoirs, which can generally be formed by one compartment each or by a section of a flow path arranged between the reagent reservoir and the barrier, are prepared with different amounts of analyte and/or a competitive antagonist of the analyte applied in each case. Preferably, different amounts of the analyte and/or a competitive antagonist of the analyte are applied parallel to the sample in at least 4 compartments in order to determine the analyte content of the sample by the signals generated in the detection area and in the control area. For determining the analyte content in the sample, the quotient can be calculated from the signal that is determined for the label of the labelled binding molecule bound in the detection area (detection signal) and the signal that is determined for the labelling of the labelled binding molecule bound in the control area (control signal):
Quotient=detection signal/control signal.

This quotient is preferably converted to a logit value as
logit value=ln(quotient/(1−quotient)), wherein, if the quotient is >1, the quotient is multiplied by a factor <1 to avoid a negative value for (1−quotient), e.g. by a factor of 0.9. The quantities of analyte applied or of the competitive antagonist of the analyte are added to a variable analyte concentration and the sum is used in the calculation as the decadic logarithm. In a numerical procedure, the analyte concentration is varied and a linear regression is performed in which the logit value is plotted against the logarithmised varied total concentration. The analyte content in the sample is determined as the minimum of the residual deviation.

FIG. 1 shows a test strip 10 which on a carrier I has fixed a porous material of five parallel flow paths 2, with the first ends 3 of which a feed material 4 is in contact through compartments 5a of a reagent reservoir 5. Opposite its compartments 5a, the reagent reservoir 5 adjoins the feed material 4. Adjacent to the opposite second ends 6 of the flow paths 2, a common absorbent material 13 is attached to the carrier 1. Optionally, a filter layer 8 is arranged between the feed material 4 and the first end 3, or a filter layer 8, e.g. a plasma separation layer, is arranged on the accessible surface of the feed material 4. Between the first end 3 and the second end 6, each flow path 2 is interrupted by a barrier 20, for example in the form of a recess 7. A detection area 9 is arranged between the barrier 20 and the second end 6 of the flow paths 2, and a control area 12 is arranged between the detection area 9 and the second end 6. Optionally, a second reagent reservoir 14 is arranged between the feed material 4 and the reagent reservoir 5, or between the reagent reservoir 5 and adjacent to the flow paths 2.

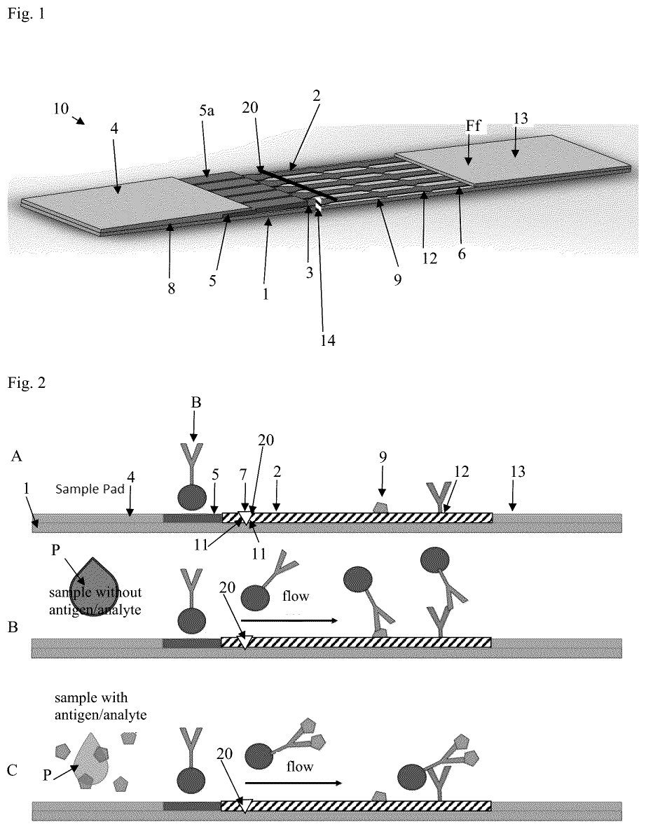

FIG. 2 schematically shows an embodiment of the method, in FIG. 2A without sample, in FIG. 2B with sample P applied to the feed material 4, which does not contain analyte A. and in FIG. 2C with sample P applied to the feed material 4, which contains analyte A. The test strip 10 shown schematically in longitudinal section has an adjacent feed material 4 at the first end 3 of the flow path 2 and a reagent reservoir 5, which connects the feed material 4 to the first end 3 of the flow path 2. The reagent reservoir 5 contains a labelled antibody as binding molecule B, e.g. an anti-AMT antibody specific for amitriptyline (AMT), which is labelled with a gold nanoparticle. A recess 7 is arranged between the first end 3 and the detection area 9 as a barrier 20, which completely interrupts the flow path 2. The analyte AMT is immobilised in the detection area 9, e.g. as a conjugate of AMT with bovine serum albumin (BSA) or nortriptyline, which is a metabolite of amitriptyline that binds to the flow path 2, in particular in the embodiment of the flow path 2 made of nitrocellulose. A control area 12 is arranged between the detection area 9 and the second end 6 of the flow path 2, which consists, for example, of a second antibody applied to the flow path 2, preferably immobilised, which is directed against the binding molecule B, for example against the heavy chain of an antibody used as binding molecule B.

After the sample has been applied to the feed material 4 and an incubation period of e.g. 3 to 4 min has elapsed, in order to open the barrier 20 the test strip 10, resp. its carrier 1, is bent along the recess 7 until the surfaces 11 of the flow path 2, which delimit the recess 7, touch, so that liquid from the feed material 4 and the reagent reservoir 5 flows through the section of the flow path 2, which extends between its first end 3 and the recess 7, across the recess 7. The flowing liquid moves the binding molecule B across the detection area 9.

A sample P applied to the feed material 4, which sample does not contain any analyte A (FIG. 2B) results in binding molecules B that do not have any bound analyte A and are therefore bound in the detection area 9 to analyte immobilised there. Binding molecules B that are not bound in the detection area 9 are moved with the flow of the liquid towards the second end 6 of the flow path 2 and can be bound in the control area 12 by the second antibody immobilised there.

For detection, the amount of label, in this example gold nanoparticles, of the binding molecule B in the detection area 9 and preferably in the control area 12 is determined, preferably measured, e.g. as colouring.

Upon application to the feed material 4 of a sample P containing the analyte A (FIG. 2 C) to the feed material 4, binding molecule B from the reagent reservoir 5 will bind the analyte A. After bending the test strip 10 about the recess 7, the barrier in this embodiment is opened and binding molecule B contained in the liquid, which has bound the analyte A, will not bind to the analyte A immobilised there when flowing through the detection area 9, but binding molecule B can be bound in the control area 12.

Optionally, a comparison of the label detected in the detection area 9 and in the control area 12 can be performed and the ratio can be used as part of the quantitative analysis.

### Example 1

### Production of Flow Paths from Nitrocellulose with a Recess Through the Nitrocellulose

Nitrocellulose on a plastic carrier made of 100 μm thick transparent polyester (type CN140, available from Sartorius, Göttingen) was used as an example of a porous material. The barrier was designed as a recess and produced by laser irradiation with a frequency-doubled Yb:KGW solid-phase laser (Light Conversion Pharos, Lithuania), wavelength 1030 nm, repetition rate 600 kHz, pulse energy 9.42 μJ, travelling speed over the nitrocellulose 2400 mm/s, 3 repetitions, laser power 5.655 W.

The flow paths were produced by ablating nitrocellulose between the flow paths to be produced with the same laser, set to a repetition rate of 600 kHz, pulse energy 11.23 μJ, travelling speed over the nitrocellulose 2400 mm/s, 3 to e.g. 20 repetitions, laser power 6.740W.

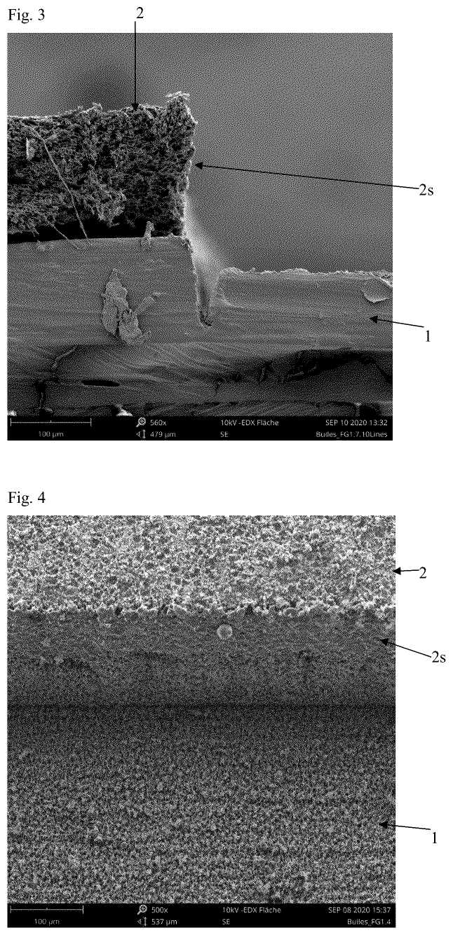

FIG. 3 shows a scanning electron micrograph (scale bar 100 μm) of the carrier 1 with a flow path 2 on it in cross-section in the area of the surface of the side wall 2s. FIG. 3 shows that the nitrocellulose of the flow path 2 has been removed right into the carrier 1 by means of laser radiation, so that the neighbouring area of the carrier 1 is free of nitrocellulose and therefore creates a distance to a further flow path. In the carrier 1 the laser irradiation has also created an incision that runs parallel to the side wall 2s. The carrier spaces apart parallel flow paths in the area where the nitrocellulose was removed, e.g. by approx. 7 μm.

FIG. 4 shows a scanning electron micrograph (scale bar 100 μm) in elevation of the carrier 1 with a flow path 2 made of nitrocellulose on it. The surface of the side wall 2s of the flow path 2 is essentially closed. This shows that by laser irradiation the porous material can be removed from the carrier 1 between flow paths 2 and a liquid-impermeable surface of the side wall 2a of the flow paths can be created.

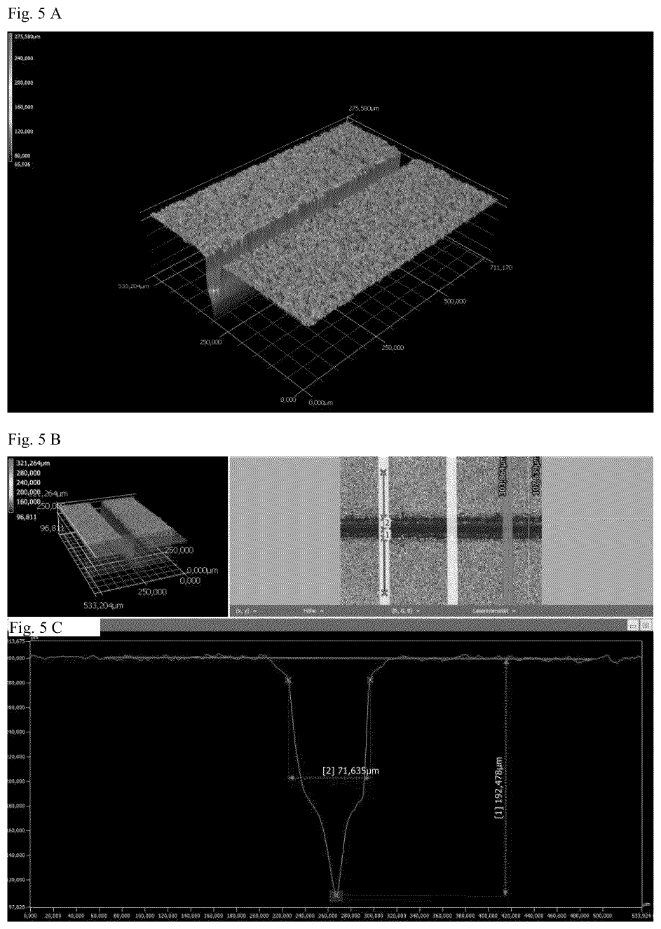

FIG. 5A-C shows scanning laser microscopic analyses of a recess 7 created by laser irradiation in a flow path made of nitrocellulose on a carrier of 100 μm thickness made of polyester. The analysis shows that the recess within the flow path adjacent to the carrier has a spacing of approx. 71 μm between the opposing surfaces 11 and extends from the surface of the flow path opposite the carrier for approx. 192 μm into the carrier.

### Example 2

### Analysing a Sample

A test strip according to FIG. 1 was used, whose flow paths 2 consisted of nitrocellulose with liquid-tight surfaces 2s of the side walls 2, as binding molecule B a murine anti-amitriptyline antibody (5 ng per flow path) with a label of gold nanoparticles (20 nm diameter), which was applied in aqueous solution to the cellulose reagent reservoir 5 and then optionally dried, with a nortriptyline-BSA conjugate as immobilised molecule, which was applied in aqueous solution as a detection area 9 to the flow paths 2 and optionally dried, with an anti-mouse antibody (from goat), which was applied in aqueous solution as a control area 12 to the flow paths 2 and optionally dried, and an absorbent material 13 made of cellulose. The flow paths 2 were preferably dried after application of the immobilised molecule and the second antibody in order to immobilise these compounds on the nitrocellulose, and then soaked with BSA (bovine serum albumin) as a non-specific protein, optionally additionally sucrose, in aqueous solution in order to saturate free binding sites of the nitrocellulose, and then dried. The reagent reservoir was preferably dried after application of the binding molecule, wherein the applied aqueous solution of the binding molecule can optionally contain a non-specific protein. Alternatively, a non-specific protein, e.g. BSA, and further optionally sucrose can be added to the sample.

As an example of a sample, aqueous phosphate buffered solution (PBS, pH 7.4) or blood plasma or whole blood, each with added amitriptyline as analyte, e.g. adjusted to a concentration of 10 ng amitriptyline/mL, was dripped onto the feed material at 200 μL. This sample dispersed into the reagent reservoir and flowed into the sections of the flow paths between their first ends and the recess.

After an incubation time of 5 min, measured after dripping the sample, the test strip was bent along the recess in order to bring the surfaces 11 of the flow paths 2, which are spaced apart by the recess 7, into contact with each other. Due to the elasticity of the carrier 1, the test strip bent back again when it was placed on a table so that it was arranged approximately in one plane. The liquid flowed through the recess along the detection areas 9 and the control areas 12 into the common absorbent material 13, as can be seen from the liquid front Ff in FIG. 1. The slight colouring visible in FIG. 1 in the detection areas 9 and the control areas 12 shows the detection of the analyte by low or absent colouring or binding of the binding molecule B, which has bound the analyte, in the detection area 9. The colouring in the control area 12 shows the binding of the portion of the binding molecule that has not bound in the detection area, regardless of the binding of the analyte.

In a further embodiment, the test strip had a second reagent reservoir 14 in the flow paths 2 between their first ends 3 and the recess 7. When the second reagent reservoir 14 was prepared by dripping on a solution of the analyte and then drying, a portion of the binding molecules were thereby bound competitively to the analyte of the sample.

### Example 3

### Analysing a Sample

A test strip with 6 parallel flow paths according to example 2 was used, in which a compartment was arranged between the reagent reservoir and the channels, of which five compartments were provided with applied analyte amitriptyline as spiking. The amounts applied were 0 pg, 5 pg, 10 pg, 20 pg, 40 pg and 80 pg amitriptyline respectively. As a sample 270 pL buffer was used, which contained no amitriptyline or a concentration of amitriptyline set to 1 μg/L or 10 μg/L.

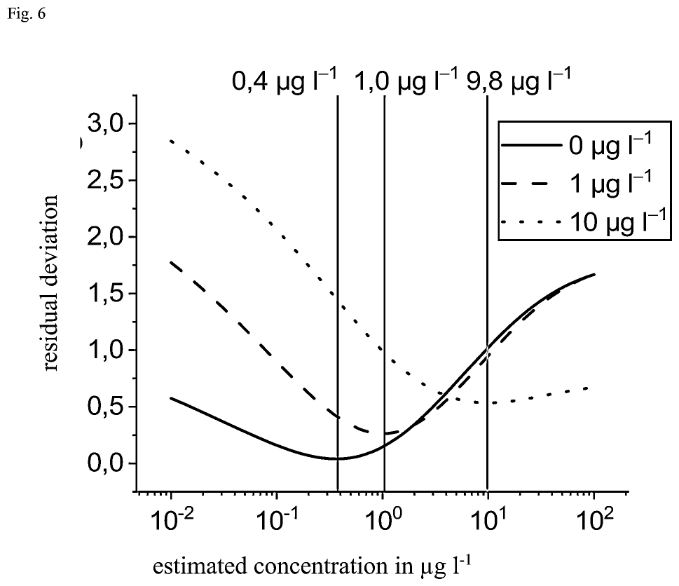

FIG. 6 shows the mathematical function in which the residual deviations are plotted for variable and freely selected analyte concentrations (labelled as guessed concentration in FIG. 6). The result shows that the minimum of the function indicates the amount of the analyte in the sample or the concentration of the analyte in the sample volume applied. The values shown above are the concentrations determined as the minimum, the concentrations of analyte used in the sample are shown on the right. Here, a value of 0.4 μg/L was determined for a sample concentration of 0, a value of 1 μg/L for a sample concentration of 1, and for a sample concentration of 10 μg/L a value of 9.8 g/L.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A test strip comprising:
at least two spaced-apart flow paths of porous material on a common carrier,
each of the at least two spaced-apart flow paths comprising a first end in contact with a feed material and a reagent reservoir adjacent to the flow paths,
a detection area in each of the at least two spaced-apart flow paths between its first end the first and its-an opposite second end, and
a control area in each of the at least two spaced-apart flow paths between the detection area and the second end, wherein the reagent reservoir is subdivided into compartments, each of which is individually connected to one of the at least two spaced-apart flow paths, wherein and each of the compartments or each of the at least two spaced-apart flow paths has comprises a controllable barrier arranged to allow the sample to flow into each of the at least two spaced-apart flow paths after opening.

2. The test strip according to claim 1, wherein at least two of the compartments contain a different amount of an analyte and/or of a competitor of the analyte, and/or a different binding molecule which is specific for a different analyte, and/or a different amount of the binding molecule.

3. The test strip according to claim 1, wherein the controllable barrier is formed as sugar, as wax, as salt, as polyvinyl alcohol, as polydimethylsiloxane, as a glass fibre mat inserted into a recess, as a section of cellulose inserted into a recess, as spiropyran-doped poly (DEAEMA-co-MMA), as a recess which can be overcome by an aqueous sample by applying an electric field, or as donor-acceptor stenhouse adducts and wax, or a combination of at least two of these.

4. The test strip according to claim 1, wherein the barrier is a recess which that extends over an entire cross-section of the compartments or of the flow path.

5. The test strip according to claim 3, wherein the recess extends into a range from 0 to 80% of a thickness of the carrier.

6. The test strip according to claim 1, wherein at least one of the compartments and/or at least one of the flow paths comprises a second reagent reservoir between the first end and the controllable barrier.

7. The test strip according to claim 1, wherein surfaces of side walls of the flow paths are impermeable to liquid.

8. The test strip according to claim 3, wherein the recess extends from a plane of a surface of the flow paths opposite the carrier with a tapered cross-section towards a plane of the carrier.

9. The test strip according to claim 3, wherein the recess extends in a plane of the surface of the flow paths opposite the carrier over a cross-section, the extent of which corresponds once to twice the thickness of the flow paths on the carrier.

10. The test strip according to claim 1, wherein the reagent reservoir has a labelled binding molecule (B) specific for an analyte (A), the detection area has an immobilised molecule which binds to the binding molecule (B) like the analyte (A), and the control area has an immobilised second antibody which is directed against the labelled binding molecule (B).

11. A method for producing a test strip according to claim 1, comprising the steps of
subdividing a porous material arranged on a carrier by laser irradiation into at least two flow paths separated from each other by a spacing, which flow paths extend from their first ends to their opposite second ends,
fusing of the surfaces of the-longitudinal sides of the flow paths to form a liquid-impermeable surface,
arranging a reagent reservoir having parallel spaced-apart compartments, with the compartments each at one of the flow paths,
producing a barrier in each of the compartments or in each of the flow paths,
arranging a reagent reservoir containing a labelled binding molecule in contact with the first end of each of the flow paths,
between the barrier and the second end of each flow path applying an immobilised molecule which binds to the binding molecule (B) like the analyte (A) to produce a detection area,
between the detection area and the second end of each flow path arranging an immobilised second antibody directed against the labelled binding molecule (B) to produce a control area, and
arranging a feed material in contact with the reagent reservoir and opposite the flow paths.

12. The method according to claim 11, wherein the reagent reservoir at its second end has compartments, each containing a different amount of an analyte and/or a competitor of the analyte, and/or a different binding molecule specific for a different analyte, and/or of a different amount of the binding molecule, one compartment each being connected to one of the flow paths.

13. The method according to claim 11, comprising applying a competitive antagonist of the analyte and/or the analyte a second reagent reservoir between the first end of the flow paths and the barrier of at least one of the flow paths.

14-17. (canceled)

18. A method for analysing a sample for the content of an analyte using a test strip according to claim 1, comprising the steps of
applying the sample to the feed material,
incubating the test strip,
opening the barrier,
flowing the sample from the reagent reservoir into the flow paths,
detecting the label of the labelled binding molecule bound in the detection area,
detecting the label of the labelled binding molecule bound in the control area.

19. The method according to claim 18, comprising opening the controllable barrier by contact with the sample or by irradiation with light, by application of an electric field, or by bending the test strip about the recesses.

20. (canceled)

21. The method according to claim 18, wherein the analyte has exactly one epitope for an antibody.

22. The method according to claim 18, wherein at least one of the flow paths comprises a second reagent reservoir with a competitive antagonist of the analyte (A).

23. The method according to claim 18, wherein between the second end of the reagent reservoir, at least four compartments are arranged to each of which a different amount of an analyte and/or a competitive antagonist of the analyte is applied, the quotient of the signal determined for the label of the labelled binding molecule bound in the detection area (detection signal) and the signal determined for the label of the labelled binding molecule bound in the control area (control signal) is determined, are calculated for each flow path as
quotient=detection signal/control signal, from which the logit values are calculated as logit value=ln(quotient/(1−quotient)), wherein in the event that quotient >1the quotient is multiplied by a factor <1 in order to avoid a negative value for (1−quotient), the logit values are plotted against the decadic logarithm of the quantities of the analyte or the competitive antagonist applied are added to a variable analyte concentration and, after a linear regression, the analyte content in the sample is determined as the minimum of the residual deviation.
